(19) United States
(12) Patent Application Publication
(10) Pub. No.: US 2026/0158092 A1
(43) Pub. Date: Jun. 11, 2026

(54) COMPOSITION COMPRISING AT LEAST ONE CANNABIS EXTRACT FOR USE IN THE TREATMENT OF AN INFLAMMATORY BOWEL DISEASE

(71) Applicant: LINNEA SA, Riazzino (Locarno) (CH)

(72) Inventors: Nora ROSSINI, Gavirate (Varese) (IT); Umberto CIRIELLO, Giubiasco (TI) (CH); Mario DELL'AGLI, Milano (IT); Valeria DE BONA, Maccagno con Pino e Veddasca (Varese) (IT); Giulia MARTINELLI, Como (IT); Giuseppe PALADINO, Seseglio (TI) (CH); Stefano PIAZZA, Seveso (Monza Brianza) (IT); Enrico SANGIOVANNI, Bergamo (IT)

(73) Assignee: LINNEA SA, Riazzino (Locarno) (CH)

(21) Appl. No.: 19/150,105

(22) PCT Filed: May 13, 2024
(86) PCT No.: PCT/EP2024/063041

§ 371 Date: Jul. 22, 2025

(30) Foreign Application Priority Data
Sep. 20, 2022 (CH) CH001032/2022
Sep. 20, 2023 (IT) 102023000019374

(51) Int. Cl. A61K 36/185; A61K 31/00; A61P 1/00; A61P 29/00

(52) U.S. Cl. CPC A61K 36/3482; A61K 31/658; A61P 1/00; A61P 29/00; A61K 2236/333

(57) ABSTRACT

A composition comprising a blend of at least two standardized extracts in their active components coming from a stable variety of Cannabis sativa L., for use in the treatment of an inflammatory bowel disease and/or in the prevention of bowel diseases correlated to acute, chronic inflammation and dysbiosis, said standardized extracts comprising in their active components cannabigerol and cannabidiol in various mass ratios.

### CROSS-REFERENCE TO RELATED APPLICATIONS

The present application is a U.S. National Phase Application under 35 U.S.C. § 371 of International PCT/EP2024/063041 filed May 13, 2024, which claims priority of Italian Patent Application No. 102023000019374 filed Sep. 20, 2023, and Swiss Patent Application No. 001032/2023 filed Sep. 20, 2023. The entire contents of which are hereby incorporated by reference.

### FIELD OF THE INVENTION

The present invention refers to a composition comprising at least one extracts of Cannabis sativa L. for use in the treatment of an inflammatory bowel disease and/or in the prevention of bowel diseases correlated to chronic inflammation and dysbiosis.

### BACKGROUND

The gastrointestinal barrier plays an important role in modulating and preventing the passage of macromolecules and pathogenic organisms, while allowing the absorption of nutritional substances, such as minerals, vitamins and other nutrients, as well as substances capable of acting on body homoeostasis and general physiological functions. The intestinal barrier is the first line that separates the inside of the body from the external environment. Proper functioning of the intestinal epithelial barrier is essential for the maintenance of general well-being and for the regulation of a number of functions, including immunity and hormonal balance.

The intestinal barrier is a complex structure, formed by highly specialized epithelial cells such as colonocytes and enterocytes. Enterocytes are intestinal epithelial cells that constitute the classic brush border at the intestinal level. Their function is mainly to promote the absorption of nutritional substances and digestion but, at the same time, to prevent the passage of xenobiotics and molecules potentially toxic to the body. Also the colonocytes, the main epithelial cells of the colon, are involved in the production of cytokines and chemokines involved in intestinal immune processes.

The plasma membrane of the intestinal epithelial cells has a variety of specialized structures that have specific barrier and transport functions. The lateral surfaces of adjacent cells are connected to each other by specialized intercellular junctions, that is, multiprotein complexes that form continuous and compact layers.

Among these, a fundamental role is played by the tight junctions: adhesive complexes that block the passage of molecules in the interstices between adjacent cells. Among the proteins that compose them, occludins and zonulins play an important role, able to connect these junctions to the cytoskeleton.

The “leaky-gut syndrome” is a syndrome characterized by the increased permeability of the intestinal mucosa, which becomes unable to act as a barrier for bacteria, fungi and allergens, with which it is constantly in contact, compromising the balance of the mucosa itself and the bacterial flora (Visser et al., 2009). The three characteristic features of this syndrome are: activation of the immune system, chronic inflammatory response and consequent increase in intestinal permeability. There are many diseases that have these three characteristics in common: some of them are, for example, fibromyalgia, ulcerative colitis, Crohn's disease and celiac disease.

The continuous activation of the immune system stimulates chronic intestinal inflammation, which in turn causes an increase in paracellular permeability; this process creates a vicious cycle that promotes a worsening of the clinical picture. Immune cells, activated at the intestinal level, migrate into the systemic circulation and reach other organs, where they can damage healthy tissues by promoting inflammatory and/or autoimmune phenomena.

The intestinal barrier, therefore, can be compromised not only by the presence of external pathogenic organisms but also due to the prolongation of an inflammatory state that can persist over time, triggering processes called chronic inflammatory bowel diseases (IBD), such as Crohn's disease or ulcerative rectocolitis.

Among the various manifestations associated with inflammatory bowel diseases are: abdominal pain, vomiting, diarrhea, blood in the stool, abundant mucus and flatulence. The prognosis of such diseases can be very unfavorable for the affected subjects and the symptoms manifest for a long time.

It is clear that gastrointestinal disorders, due to the presence of numerous toxins of an infectious or inflammatory nature (or both), are debilitating diseases.

Leaky-gut syndrome seems to be correlated to the onset of chronic-degenerative diseases such as Parkinson's disease and fibromyalgia.

For example, protein aggregates have been observed at the intestinal level in the prodromal stages of Parkinson's disease, often associated with dysbiosis. Dysbiosis consists of an alteration of the intestinal flora and is promoted and often caused by the abuse of drugs (antibiotics, antifungals, cortisones, NSAIDs, etc.) and by conditions of inflammatory alteration of the intestinal mucosa that prevent the adhesion of eubiotics and their protective function of body homeostasis. In fact, the bacterial flora has the important role, together with the intestinal barrier, of digesting and metabolising many molecules, neutralizing toxins and allowing the use of many nutrients, such as short-chain fatty acids. These molecules are precursors to many neurotransmitters and their deficiency is responsible for alterations in brain immune response and microglial signaling.

In a context of dysbiosis, characterized by an increased permeability of the intestinal epithelium subjected to inflammatory phenomena, the probability of neurotoxins passing through the intestinal barrier is increased and the presence of intestinal inflammation, as well as inflammation of the enteric nervous system, can be accompanied by symptoms no longer localized in the intestine but which extend to other districts and organs, including the central nervous system.

Similarly, patients with fibromyalgia have alterations in the bacterial flora of the small intestine (small intestine bacterial overgrowth or SIBO). The increase in intestinal permeability seems to be correlated with the onset of fibromyalgia disease and the impairment of peripheral serotonergic transmission, primarily modulated at the intestinal level, potentially explaining the onset of painful symptoms.

Gastrointestinal disorders, especially chronic ones, often require lifelong treatments and above all require specific care to manage the symptoms that can disrupt the daily lives of the affected people.

The treatments in use today are non-specific and often with limited effectiveness, both because it is difficult to diagnose disorders of the gastrointestinal tract, which often have very similar symptoms to each other, and because the drugs in use do not always generate the desired response or the response is not compensated by adequate compliance, especially on long-term treatments. For example, the administration, both orally and rectally, of aminosalicylates and analogues, such as 5-Aminosalicylic acid (5-ASA). This drug acts on the mucosal epithelial cells by inducing a reduction in the release of inflammatory mediators (i.e. prostaglandins E2, thromboxane and leukotrienes).

Many side effects are known from the use of aminosalicylates and analogues, including a potential risk of nephrotoxicity. Patients treated with these drugs should closely monitor renal functionality prior to initiation of therapy and periodically during therapy.

Another class of drugs used to treat ulcerative colitis or Crohn's disease are corticosteroids.

Approximately 50% of patients treated with systemic corticosteroids have side effects related to significant doses, medium-long term therapy or related to discontinuation.

Another class of drugs used in both ulcerative colitis and Crohn's disease are thiopurines (i.e. Azathioprine and 6-mercaptopurine) which exert their immunosuppressive activity by modulating T lymphocyte apoptosis.

The most frequent side effects of thiopurines are medullary depression, hepatitis, pancreatitis, allergic reactions, fever, vomiting, diarrhea, increased infectious risk, including also opportunistic infections, particularly when the drug is associated with the steroid.

An increase in the risk of non-melanoma skin neoplasms is also documented in patients treated with thiopurines.

### SUMMARY

The present invention intends to provide an alternative composition with respect to those already on the market, to be used in the treatment of an inflammatory bowel disease and which allows to solve the drawbacks described in the prior art.

Within the scope of this technical task, an object of the present invention is to identify a composition having a high anti-inflammatory activity.

Another object of the present invention is to provide a composition for use in the treatment of an inflammatory bowel disease which exhibits a high inhibitory capacity against inflammation and which at the same time exhibits no toxicity at the cellular level.

A further object of the present invention is to provide a composition for use in the treatment of an inflammatory bowel disease which is particularly effective at low doses and which has no side effects.

A further object of the present invention is to provide a composition for use in the treatment of an inflammatory bowel disease that is reproducible and constant over time to allow a more effective treatment.

Finally, the present invention aims to provide a composition to be used in the treatment of an inflammatory bowel disease characterized by dysbiosis and alteration of permeability, since the products to be protected are capable of improving intestinal permeability.

These and other objects of the present invention are achieved by a composition comprising extracts of Cannabis sativa for use in the treatment of an inflammatory bowel disease.

Other salient aspects of the invention are set forth in the following dependent claims.

### BRIEF DESCRIPTION OF THE DRAWINGS

Further characteristics and advantages of the invention will become more apparent from the description of some preferred, but not exclusive embodiments of the composition comprising extracts of cannabis for use in the treatment of an inflammatory bowel disease, which is illustrated by way of non-limiting example in the attached drawings, of which:

FIG. 1: evaluation of gene expression of alpha1-antitrypsin (AIAT), sucrase-isomaltase (SI), apolipoprotein C-3 (APOC3) and apolipoprotein A-1 (APOA1) by real-time RT-PCR in CaCo-2 cells (colonocytes) before and after 17 days of differentiation to enterocytes.

FIG. 2: evaluation of cell viability (MTT) of the enterocytes following treatment with extracts of C. sativa L. at 100 μg/mL for 24 hours, concomitantly with stimulation by means of a pro-inflammatory cocktail consisting of 50 ng/ml TNFα, 10 ng/mL LPS, 50 ng/ml IFNγ and 25 ng/mL IL-Iβ. The results were reported as a percentage with respect to the pro-inflammatory stimulation. 50 μM EGCG (Epigallocatechin gallate) was used as a positive control.

FIG. 3: evaluation of transepithelial electrical resistance (TEER) in enterocytes treated with the pro-inflammatory mixture (50 ng/ml TNFα, 10 ng/ml LPS, 50 ng/mL IFNγ and 25 ng/ml IL-1B) and extracts of C. sativa L. at 100 μg/mL after 24 hours of treatment. *** p<0.001.

FIG. 4: evaluation of pro-inflammatory gene expression in enterocytes treated with the pro-inflammatory mixture (50 ng/mL TNFα, 10 ng/mL LPS, 50 ng/ml IFNγ and 25 ng/ml IL-1β) for 24 hours.

FIG. 5: evaluation of CXCL10 secretion and its modulation following treatment with the phytocannabinoids CBD and CBG compared to two pro-inflammatory stimulations, a first mixture consisting of 50 ng/ml TNFα, 10 ng/ml LPS, 50 ng/mL IFNγ and 25 ng/mL IL-1B and a second mixture consisting of 10 ng/ml IFNγ and 10 ng/ml IL-1B. 50 μM Epigallocatechin gallate (EGCG) was used as a positive control. The results were reported as a percentage with respect to the pro-inflammatory stimulation. * P<0.05, ** P<0.01, and *** P<0.001.

FIG. 6: evaluation of cell viability of colonocytes following treatment with extracts of C. sativa L. (100 μg/mL) and the individual phytocannabinoids (8 μM) for 24 hours, concomitantly with pro-inflammatory stimulation of 10 ng/ml IFNγ and 10 ng/ml IL-Ip. The results were reported as a percentage with respect to the pro-inflammatory stimulation. 50 μM Epigallocatechin gallate (EGCG) and 2 mM butyrate (SCFA) (=176.2 μg/mL) were used as positive controls.* p<0.05, ** p<0.01, *** p<0.001.

FIG. 7: evaluation of CXCL10 secretion with extracts of C. sativa L. (100 μg/mL) and cannabinoids (8 μM) at 24 hours, concomitantly with pro-inflammatory stimulation of 10 ng/ml IFNγ and 10 ng/mL IL-1B. 50 μM Epigallocatechin gallate (EGCG) and 2 mM butyrate (SCFA) (=176.2 μg/mL) were used as positive controls. The results were reported as a percentage with respect to the pro-inflammatory stimulation. * P<0.05, ** P<0.01, and *** P<0.001.

FIG. 8: evaluation of NF-KB-guided transcription in the presence of extracts of C. sativa at 100 μg/mL and cannabinoids at 8 μ Mat 6 hours, concomitantly with pro-inflammatory stimulation of 10 ng/mL IFNγ and 10 ng/ml IL-1p. 2 mM butyrate (SCFA) (=176.2 μg/mL) was used as a positive control. The results were reported as a percentage with respect to the pro-inflammatory stimulation.* p<0.05, ** p<0.01, *** p<0.001.

FIG. 9: evaluation of enterocyte cell viability following treatment with extracts of C. sativa L. (100 μg/mL) and cannabinoids (8 μM) for 24 hours, concomitantly with pro-inflammatory stimulation of 10 ng/mL IFNγ and 10 ng/ml IL-1β. The results were reported as a percentage with respect to the pro-inflammatory stimulation. 2 mM butyrate (SCFA) (=176.2 μg/mL) was used as a positive control.

FIG. 10: evaluation of transepithelial electrical resistance in enterocytes treated with the pro-inflammatory mixture (10 ng/ml IFNγ and 10 ng/ml IL-1β) in the presence of extracts of C. sativa L. (100 μg/mL) and phytocannabinoids (8 μM) for 24 hours. 2 mM butyrate (SCFA) was used as a positive control.** p<0.01, *** p<0.001.

FIG. 11: evaluation of CXCL10 secretion with extracts of C. sativa L. at 100 μg/mL and cannabinoids at 8 μMat 24 hours, concomitantly with pro-inflammatory stimulation of 10 ng/mL IFNγ and 10 ng/ml IL-1β. The results were reported as a percentage with respect to the pro-inflammatory stimulation. * p<0.05, ** p<0.01.

FIG. 12: Quantitative LC-MS analysis of CBD and CBG in extracts 5 and 6 subjected to simulated gastro-intestinal digestion. ** p<0.01 (t test). The data are the mean±SD of three injections.

FIGS. 13A and 13B: Evaluation of cytotoxicity of the extracts (5 and 6) and respective cannabinoids (CBD, CBG) by MTT assay. Concomitant treatment of extracts (100 μg/mL) or cannabinoids (8 μM) with IL-1β/IFN-γ in enterocytes is reported on the left (FIG. 13A), while treatment of colonocytes is reported on the right (FIG. 13B). *** p<0.001 (one-way ANOVA test) vs IL-1β/IFN-γ. The data are the mean±SEM of at least three duplicate experiments.

FIG. 14: Profile of pro-inflammatory response induced by IL-1B (10 ng/mL) and IFN-γ (10 ng/mL) in the colonocyte (PCR array). The general transcriptional profile by heat-map is reported on the left, while the genes significantly up-regulated (CCR6, CSF1, CXCL9) or down-regulated (AIMP1, CCL15) with respect to the unstimulated control are listed on the right.

FIG. 15A and 15B: Profile of pro-inflammatory response induced by IL-1β (10 ng/mL) and IFN-γ (10 ng/mL) in the colonocyte, following treatment with the extracts 5 and 6 (100 μg/mL) subjected to simulated intestinal digestion (5d, 6d). The general transcriptional profile by heat-map is reported above, while the genes significantly up-regulated (NAMPT) or down-regulated (CXCL-9) by the treatment with respect to the pro-inflammatory stimulation are listed below.

FIGS. 16A and 16B: Profile of the pro-inflammatory response induced by IL-1β (10 ng/ml) and IFN-γ (10 ng/ml) in the enterocyte (PCR array). The general transcriptional profile by heat-map is reported in FIG. 16A, while the up-regulated genes with respect to the unstimulated control are listed in FIG. 16B.

FIGS. 17A, 17B, 17C and 17D: Profile of the pro-inflammatory response induced by IL-1β (10 ng/ml) and IFN-γ (10 ng/ml) in the enterocyte, following treatment with the extracts 5 and 6 (100 μg/mL) subjected to simulated intestinal digestion (5d, 6d). The general transcriptional profile by heat-map is reported, from which no significant effects of the treatment emerge. In fact, the transcriptional levels are modulated within restricted values with respect to the pro-inflammatory stimulation.

FIGS. 18A, 18B, 18C and 18D: Effect of the extracts 5 and 6 subjected to intestinal digestion (5d, 6d) and of the cannabinoids (CBD, CBG) on the release of inflammatory mediators (CXCL-9, CXCL-10, CCL-20, IL-15) by the colonocyte. The extracts (100 μg/mL) and the isolated molecules (8 μM) were used in the treatment concomitant with pro-inflammatory stimulation (IL-1β/IFN-γ), arbitrarily placed at 100% . * p<0.05; ** p<0.01; *** p<0.001 (one-way ANOVA test) vs IL-1β/IFN-γ. The data are the mean±SEM of at least three duplicate experiments.

FIG. 19: Effect of the extracts 5 and 6 subjected to intestinal digestion (5d, 6d) and of the cannabinoids (CBD, CBG) on the release of inflammatory mediators (CXCL-9, CXCL-10) by the enterocyte. The extracts (100 μg/mL) and the isolated molecules (8 μM) were used in the treatment concomitant with pro-inflammatory stimulation (IL-1β/IFN-γ), arbitrarily placed at 100% . * p<0.05; ** p<0.01; *** p<0.001 (one-way ANOVA test) vs IL-1β/IFN-γ. The data are the mean±SEM of at least three duplicate experiments.

FIGS. 20A and 20B: Effect of the extracts 5 and 6 subjected to intestinal digestion (5d, 6d) and of the cannabinoids (CBD, CBG) on epithelial permeability (enterocyte). In FIG. 20A the extracts (100 μg/mL) and the isolated molecules (8 μM) were used in the treatment concomitant with pro-inflammatory stimulation with IL-1β/IFN-γ. In FIG. 20B the extracts (100 μg/mL) and the isolated molecules (8 μM) were used in the co-culture of enterocyte and macrophage (THP-1) stimulated with LPS/IFN-γ. Pro-inflammatory stimulation is arbitrarily set to 0 to highlight positive transepithelial resistance changes (TEER, Ohm). Butyrate (2 mM) is used as a known protective SCFA for the intestinal epithelium. * p<0.05; ** p<0.01; *** p<0.001 (one-way ANOVA test) vs pro-inflammatory stimulation. The data are the mean±SEM of at least three duplicate experiments.

FIG. 21: Effect of the extracts 5 and 6 subjected to intestinal digestion (5d, 6d) and of the cannabinoids (CBD, CBG) on paracellular permeability of the fluorescent probe LY (enterocyte). The extracts (100 μg/mL) and the isolated molecules (8 μM) were used in the co-culture of enterocyte and macrophage (THP-1) stimulated with LPS/IFN-γ. The pro-inflammatory stimulation is arbitrarily set to 1. * p<0.05 (one-way ANOVA test) vs pro-inflammatory stimulation. The data are the mean±SEM of at least three duplicate experiments.

FIG. 22: Effect of the extracts 5 and 6 subjected to intestinal digestion (5d, 6d) and of the cannabinoids (CBD, CBG) on the expression of proteins, such as occludin (OCC) and zonulin (ZO-1), in the enterocyte. The extracts (100 μg/mL) and the isolated molecules (8 μM) were used in the co-culture of enterocyte and macrophage (THP-1) stimulated with LPS/IFN-γ. In the figure, the nuclei and the fluorescent interspaces between the nuclei indicative of the localization and expression of the protein under study are highlighted.

FIG. 23: Prebiotic effect of the extracts 5 and 6 subjected to intestinal digestion (5d, 6d) and of the cannabinoids (CBD, CBG) in terms of induction of butyric acid (SCFA) production by the human microbiota of fecal origin. The result is expressed in pg/mL (ELISA assay). The data are the mean±SEM of at least three duplicate experiments.

### DETAILED DESCRIPTION

The present invention concerns a composition comprising at least one cannabis extract, to be used in the treatment of an inflammatory bowel disease and/or in the prevention of bowel diseases correlated to chronic inflammation and dysbiosis.

In particular, at least one extract comes from a stable variety of Cannabis sativa L. At least one type of extract contained in the invention is standardized and titrated, i.e., as defined in the European Pharmacopoeia, it has a defined content of one or more constituents having known biological and/or therapeutic activity.

Standardization allows reproducibility to be maintained and, consequently, the treatment based on standardized extracts is constant and reproducible over time.

Preferably, at least one standardized extract comprises one of the phytocannabinoids cannabigerol and cannabidiol.

In particular, the composition comprises a blend of at least two standardized extracts coming from varieties of Cannabis sativa L., wherein said extracts comprise cannabigerol and cannabidiol in various mass ratios.

Preferably the mass ratios are chosen from I:1, I:2 or 2:1.

According to a first embodiment of the invention, the composition has cannabidiol and cannabigerol, each in a concentration equal to 5% by weight of the composition.

According to another embodiment of the invention, the composition has cannabidiol and cannabigerol respectively in concentrations equal to 5% and 2.5% or vice versa.

According to another embodiment of the invention, the composition has cannabidiol and cannabigerol, each in a concentration equal to 2.5% by weight of the composition.

Preferably the composition further comprises an MCT oil-based carrier, the function of which is to standardize the composition. MCT does not exhibit any interfering activity with the assays used in the present invention.

In particular, the inflammatory bowel disease is selected from the group comprising chronic intestinal inflammations, leaky-gut syndrome, dysbiosis, celiac disease, intestinal inflammation of bacterial or parasitic origin, diverticulitis and irritable bowel syndrome.

In particular, the chronic intestinal inflammation comprises ulcerative colitis and Crohn's disease.

According to one aspect of the invention, the standardized extracts reduce the secretion of CXCL10, a chemokine upregulated by IFNγ and with a role in attracting and activating T cells at the intestine level; this chemokine is involved in several inflammatory diseases.

According to another aspect of the invention, the composition subject-matter of the invention for use in the treatment of an inflammatory bowel disease and/or in the prevention of bowel diseases correlated to chronic inflammation and dysbiosis is administered orally.

The present invention will now be further illustrated by the following examples, which are not intended to be limiting.

### Materials and Methods

The production of extracts of cannabis is carried out following three main points:
1) Solvent extraction of different varieties of Cannabis sativa where the most abundant cannabinoid is CBD or CBG.
2) Purification of the extracts relative to the previous point by filtration, 1/1 extraction and carrier addition.
3) Mixing of extracts obtained from the previous points, with the possibility of adding additional carriers to obtain the desired concentration of the two main cannabinoids (CBD and CBG).

For the realization of point 1, it is possible to use different qualities of Cannabis sativa L. that allow to obtain crude extracts containing many components of the cannabis. The use of selected and stable varieties of cannabis allows to obtain crude extracts with constant content of the extracted and desired components. For the present invention, varieties capable of expressing high concentrations of CBG or CBD have been considered.

The choice of the extraction solvent is equally important to ensure the selectivity of the components to be extracted; organic solvents such as ethanol, methanol, pure acetone or in admixture with water or solvents such as ethyl acetate, hexane, heptane, supercritical carbon dioxide and others can be used.

Point 2 of purification allows to eliminate the components not necessary to the extract; depending on the choice of the solvent of point 1, it is possible to perform a filtration of the components not soluble in the carrier (if a pure solvent is used as extraction solvent) or an 1/1 extraction with non-polar organic solvents such as: hexane, heptane, cyclohexane, etc. (if organic solvent mixtures and water are used). The type of the extraction solvent is fundamental for the quality and activity of the extract, see phase 2 and following ones which highlights some differences in the extracts used. The use of hydroalcoholic solutions followed by 1/1 purifications with hexane allows, for example, to optimize the extract purification process, consequently amplifying its pharmacological activity.

The addition of the carrier finally allows extract pre-standardization.

Point 3 provides for the mixing and standardization of the extracts relative to point 2, typically two pre-standardized extracts are mixed in which one has a high concentration of CBD and the other a high concentration of CBG. The mixing and possible addition of carriers such as MCT oil (medium chain triglycerides), corn oil, olive oil etc., allows the desired concentration of the two cannabinoids to be obtained. For example, 1:1, 2:1, or 1:2.

The present invention summarizes the results of the scientific evaluations on the anti-inflammatory activity of the extracts of Cannabis sativa L. (C. sativa) described below in human intestinal cells. The objective of the project is to identify the extracts that show greater anti-inflammatory activity for further study. The extracts under analysis are described in the following Table 1.

TABLE 1
List of the purified cannabinoids (samples 1 and 2) and of the
extracts of C. sativa (3-12) analysed in the first phase.

| No. | Type | Batch |
|---|---|---|
| 1 | Solid CBD | CBDl 22004 |
| 2 | Solid CBG | CBG R0322038-1 |
| 3 | CM05 C. sativa CBD 5%; | CGC R0122023-4 |
| | absolute Ethanol extract | |
| 4 | CM05 C. sativa CBD 5%; | CGC R0122024-4 |
| | Ethanol extract | |
| 5 | Mix CBD/CBG 5:5%; | CGC R0122023-1 |
| | absolute Ethanol extract | |
| 6 | Mix CBD/CBG 5:5%; | CGC R0122024-1 |
| | Ethanol extract | |
| 7 | Mix CBD/CBG 5:2.5%; | CGC R0122023-2 |
| | absolute Ethanol extract | |
| 8 | Mix CBD/CBG 5:2.5%; | CGC R0122024-2 |
| | Ethanol extract | |
| 9 | Mix CBD/CBG 2.5:5%; | CGC R0122023-3 |
| | absolute Ethanol extract | |
| 10 | Mix CBD/CBG 2.5:5%; | CGC R0122024-3 |
| | Ethanol extract | |
| 11 | CG05 C. sativa CBG 5%; | CG05 R0122005 |
| | absolute Ethanol extract | |
| 12 | CG05 C. sativa CBG 5%; | CG05 R0122006 |
| | Ethanol extract | |

Absolute Ethanol extract refers to the use of pure ethanol during the phase of extraction of the cannabis raw material, while Ethanol extract refers to the use of a mixture of ethanol and water during the cannabis extraction phase.

Since bowel diseases affect different areas of the organ, both small, mainly characterized by the presence of enterocytes, and large ones, mainly characterized by colonocytes, the inventors evaluated the extracts in both cellular contexts, in order to identify the best site of application.

### Phase 1

### Example 1: Evaluation of the Anti-Inflammatory Activities of Standardized Extracts of Cannabis sativa L. In Cannabigerol (CBG) and Cannabidiol (CBD) and of the Relative Purified Cannabinoids in Undifferentiated Cells (Colonocytes)

Firstly, the individual cannabinoids CBD and CBG in undifferentiated cells (colonocytes) were analysed by comparing two different pro-inflammatory mixtures, in order to simplify the pro-inflammatory stimulation and the activation pathways involved. The pro-inflammatory mixture 50 ng/mL TNFα, 10 ng/mL LPS, 50 ng/ml IFNγ and 25 ng/ml IL-1ß mimics an inflammatory disease with a bacterial aetiology and was compared with a less complex mixture, consisting of 10 ng/mL IFNγ and 10 ng/mL IL-1β, deprived of the “bacterial” component, therefore more characteristic of an advanced inflammatory disease.

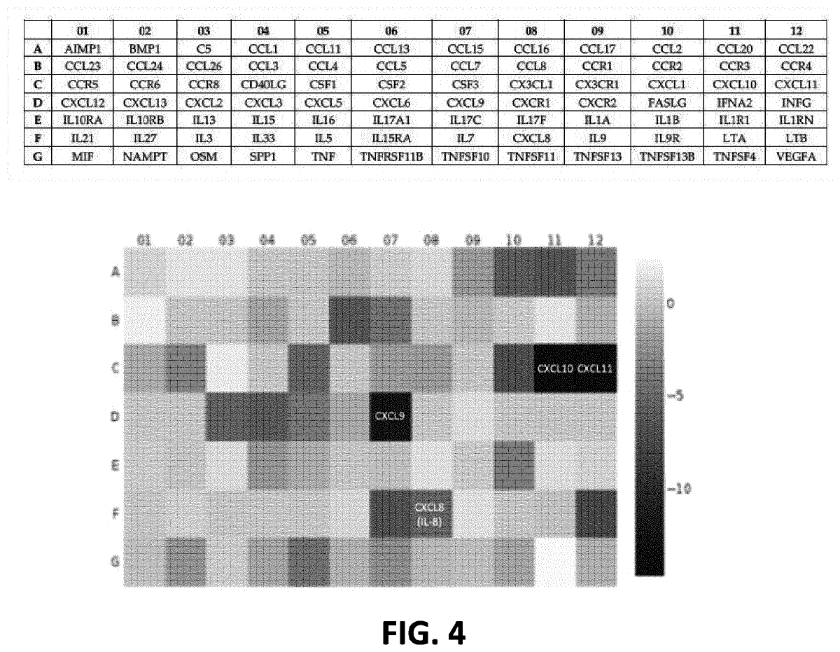

From the analysis of the pro-inflammatory genes most regulated by the application of the pro-inflammatory mixture, chemokines of the CXC family particularly over-expressed in the enterocyte and colonocyte emerged (FIG. 4). In particular, CXCL-10 is a chemokine upregulated by IFNγ, with a role in attracting and activating T cells at the intestinal level involved in several inflammatory diseases.

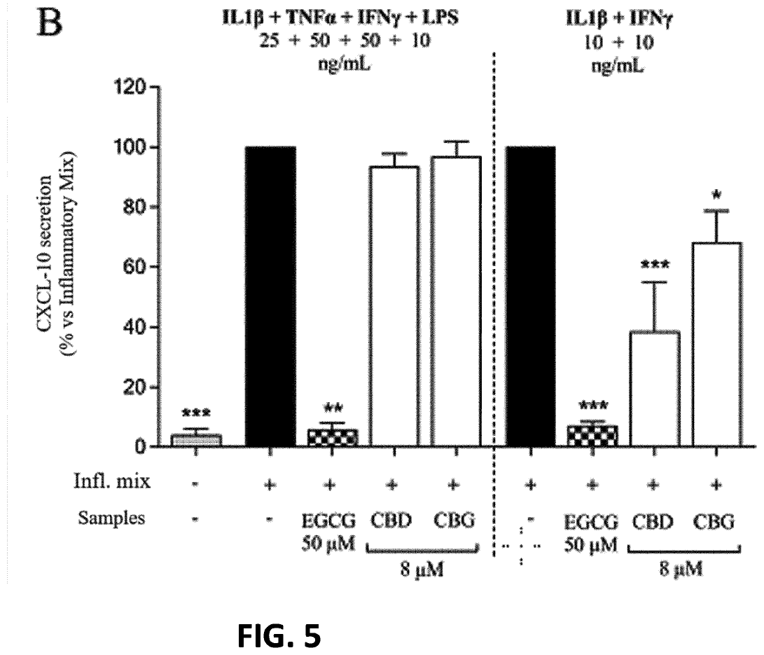

From the results it emerged that both stimulations induce the release of CXCL10 (FIG. 5) in undifferentiated cells (colonocytes).

The used concentration of the cannabinoids (8 μM) corresponds to a quantity equal to 5% in the extracts at a concentration of 100 μg/mL. Both cannabidiol (CBD) and cannabigerol (CBG) reduced CXCL10 secretion, but only with the application of the simplified pro-inflammatory mixture (10 ng/mL IFNγ and 10 ng/mL IL-1β). CBD was found more active than CBG (FIG. 5).

Based on the results obtained, the effects of the different extracts were evaluated using the pro-inflammatory stimulation consisting of 10 ng/mL IFNγ and 10 ng/mL IL-1β.

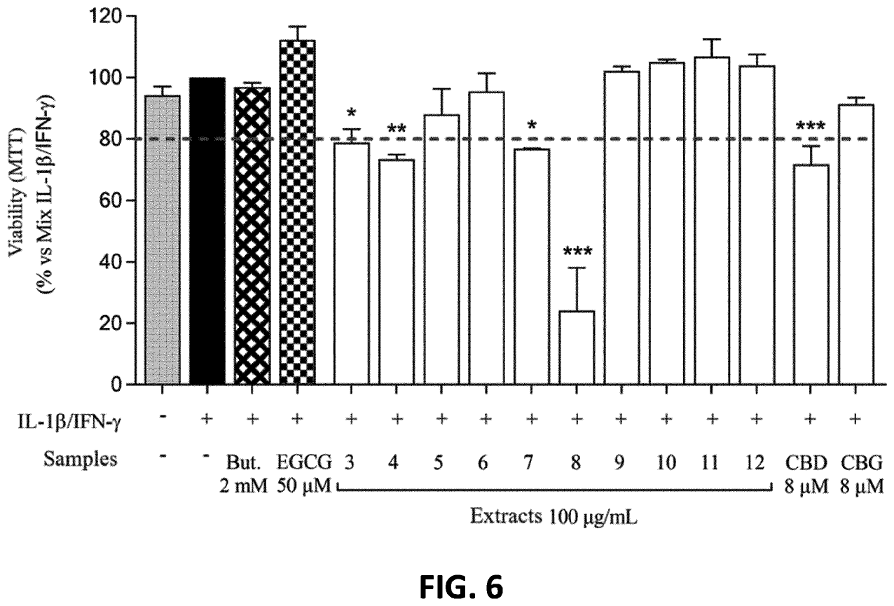

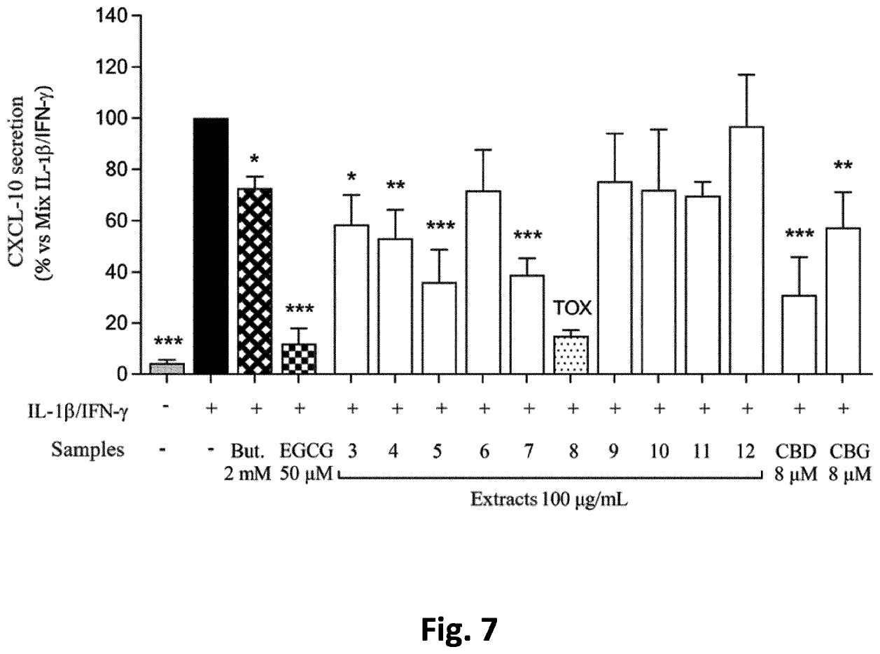

Firstly, the cytotoxicity of the extracts in the undifferentiated CaCo-2 cells (colonocytes) at 100 μg/mL was evaluated. The analysis showed a slight reduction in cell viability for the extracts 3, 4 and 7, as well as for cannabidiol at 8 μM (FIG. 6). The effect of the extract 8 is not to be considered for this model.

The extracts 3, 4, 5 and 7 produced a significant reduction in CXCL10, in particular the extracts 5 and 7, although only the former was found to be completely devoid of toxicity at this concentration; In order to investigate a possible mechanism of action, the colonocytes were transfected with a reporter plasmid containing the luciferase gene under the control of a promoter responsive to the NF-κB transcription factor. The use of this promoter allows the effect of the extracts/active molecules on transcription driven by this transcription factor to be observed.

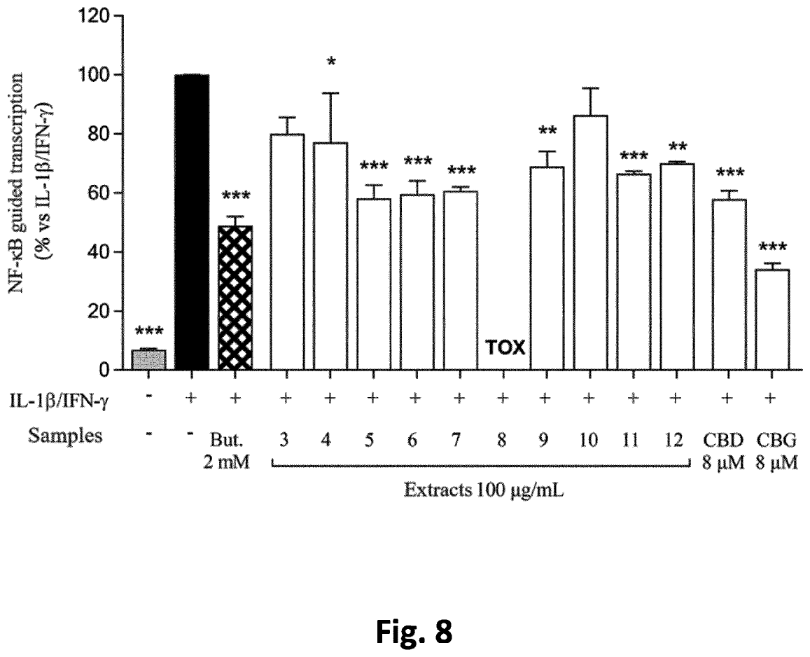

In this experiment all the extracts, except for number 3 and 10, reduced NF-KB driven transcription in a statistically significant manner. For this particular parameter, CBG was found more active than CBD (FIG. 8).

The effect on NF-κB is in line with what our group highlighted in other cell types, such as human HaCaT keratinocytes (DOI: 10.1002/ptr.6400), where an inhibitory effect on this transcription factor of both an extract of Cannabis and pure CBD at micromolar concentrations was highlighted. From the analysis of the undifferentiated cells (colonocytes), the extract 5 (CBD/CBG mix 5:5%; absolute Ethanol extract) was found to be the best in terms of inhibitory capacity and simultaneous absence of toxicity.

### Example 2: Evaluation of the Anti-Inflammatory Activities of Standardized Extracts of Cannabis Sativa L. In Cannabigerol (CBG) and Cannabidiol (CBD) and the Relative Purified Cannabinoids in Differentiated Cells (Enterocytes)

This phase of the project involved the evaluation of derivatives of Cannabis sativa L. on the basis of the anti-inflammatory activity shown in CaCo-2 cells (colonocytes) differentiated into enterocytes.

Differentiation of the CaCo-2 cells (colonocytes) into enterocytes was confirmed through gene expression of specific differentiation markers: alpha1-antitrypsin (A1AT), sucrase-isomaltase (SI), apolipoprotein C-3 (APOC3), and apolipoprotein A-1 (APOA1).

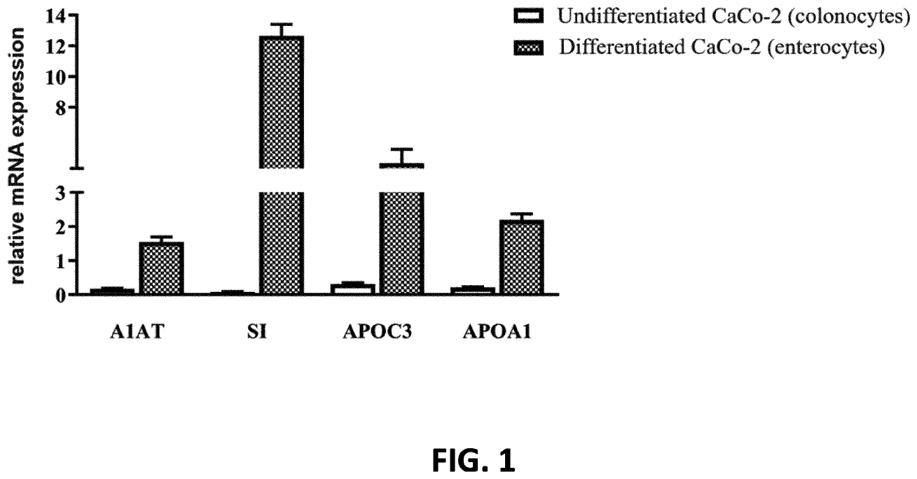

As can be observed in FIG. 1, all four genes are expressed in CaCo-2 cells differentiated into enterocytes and poorly expressed or absent in colonocytes, confirming the adequacy of the chosen cell model.

Once the differentiation was confirmed, the cytotoxicity of the extracts on the enterocytes was analysed in Transwell® plates. The cells were treated with the extracts concomitantly with the pro-inflammatory stimulation, consisting of a mixture of 50 ng/mL TNFα, 10 ng/mL LPS (E. coli lipopolysaccharide), 50 ng/mL IFNγ and 25 ng/ml IL-1β, for 24 hours. All extracts were assayed at the concentration of 100 μg/mL.

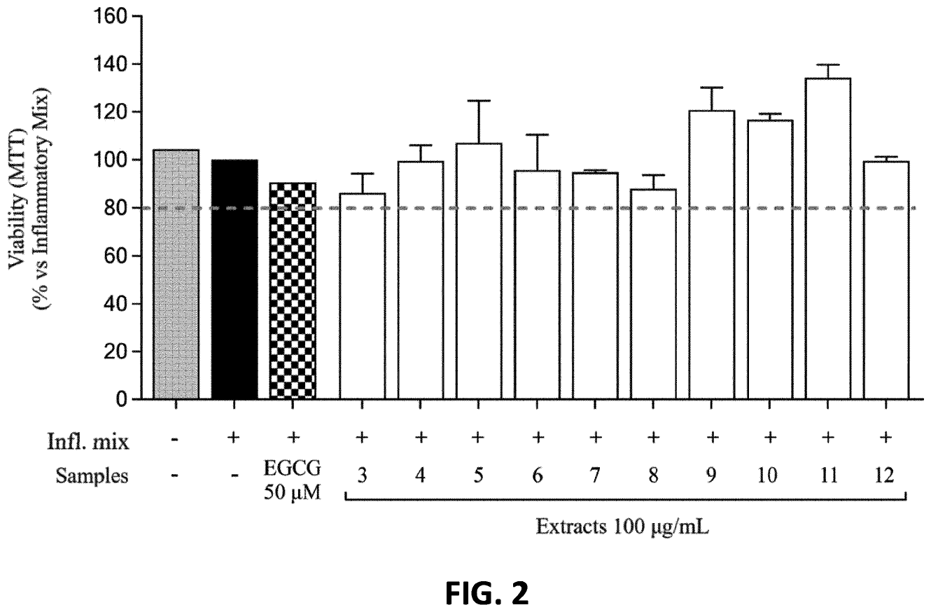

The results, visible in FIG. 2, show that none of the extracts is able to reduce cell viability.

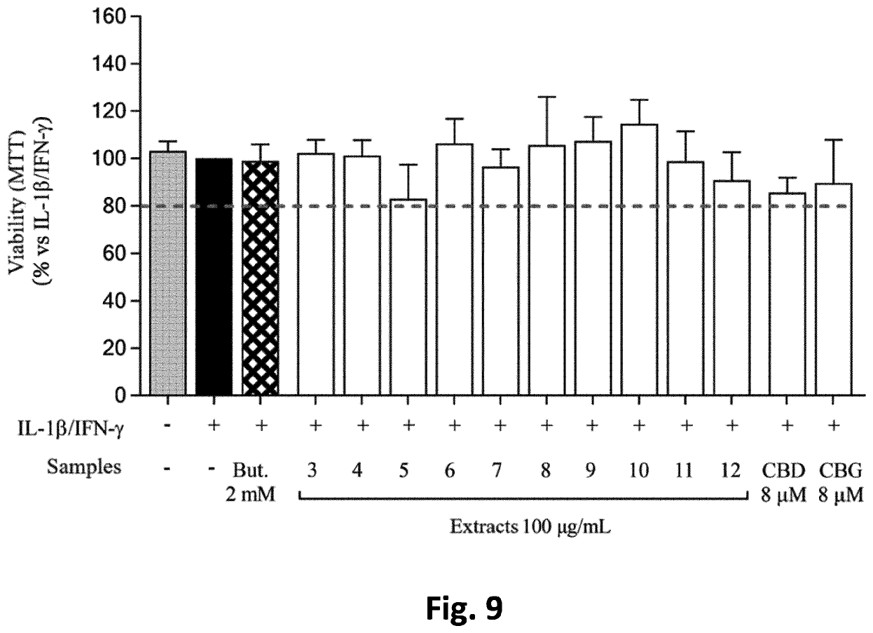

The analysis of the cytotoxicity of the extracts of C. sativa in the enterocytes, following the use of the simplified stimulation of 10 ng/ml IFNγ and 10 ng/ml IL-1β, did not lead to different results compared to those obtained with the first pro-inflammatory mixture. All the extracts (100 μg/mL) and the cannabinoids (8 μM), showed no reductions in cell viability after 24 hours, as can be observed in FIG. 9.

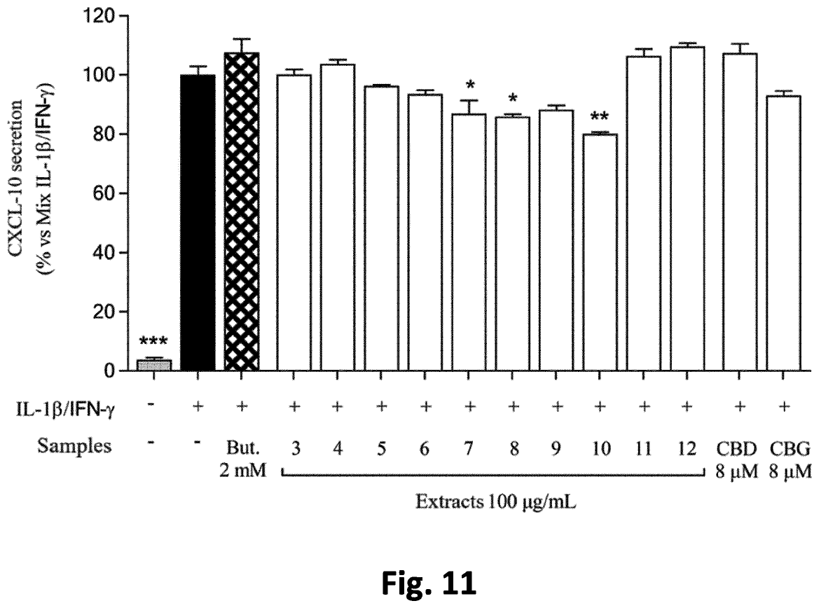

The analysis of the extracts on the reduction ofCXCL10 induced by 10 ng/ml IFNγ and 10 ng/ml IL-1B showed a significant reduction for the extracts 7, 8 and 10, even better than butyrate, used as a positive control, and no effect for the other extracts or the individual cannabinoids (FIG. 11).

### Example 3: Evaluation of the Trans-Epithelial Electrical Resistance (Teer) on Enterocytes Subjected to Inflammatory Stimulation

In order to investigate further regulation mechanisms operated by the extracts subject-matter of the present invention on this cellular model, a screening was performed on their ability to restore the transepithelial electrical resistance (TEER), as a parameter indicative of a damage to the intestinal epithelial barrier. The cells were treated for 24 hours with the pro-inflammatory stimulation (complete MIX) concomitantly with the extracts.

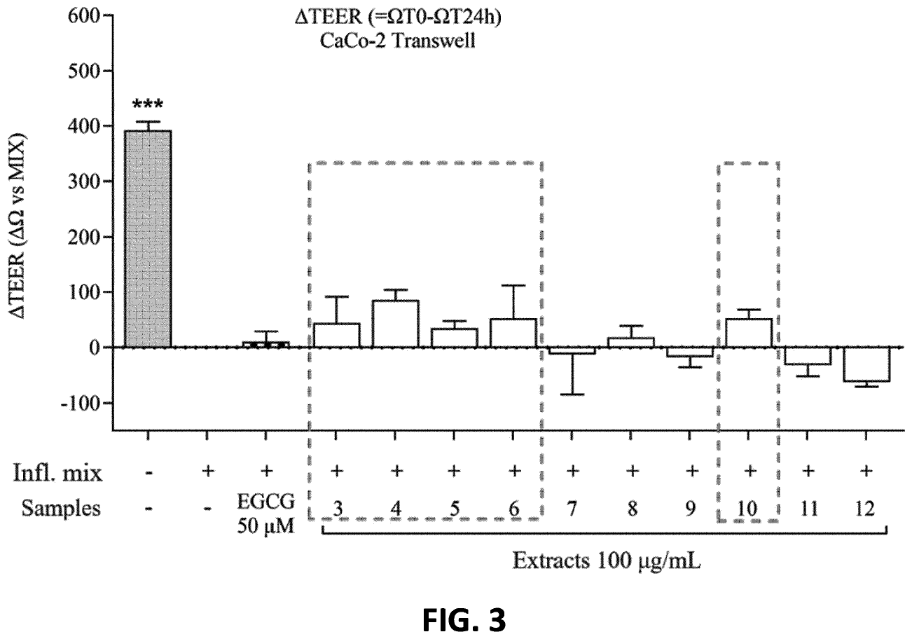

As visible from the results shown in FIG. 3, this pro-inflammatory stimulation reduces the TEER value compared to the unstimulated control cells, indicating a damage and thus an increased permeability of the cell monolayer. The extracts 3, 4, 5, 6 and 10, at a concentration of 100 μg/mL, were shown able to preserve, at least in part, the integrity of the epithelial barrier.

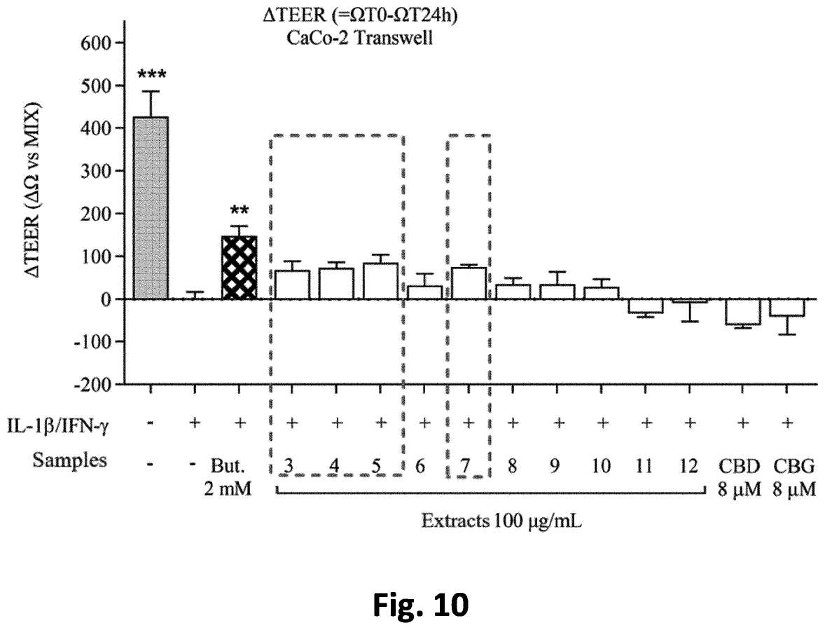

The analysis of the transepithelial electrical resistance performed after stimulation with the reduced mixture, consisting of 10 ng/ml IFNγ and 10 ng/ML IL-1β (deprived of the “bacterial” component), showed that all extracts induce an improvement of this parameter when evaluated at 100 μg/mL for 24 hours, with the exception of the extracts 11 and 12 and of the individual cannabinoids CBD and CBG. The extracts 3, 4, and 5 confirmed their ameliorative activity compared to the previous pro-inflammatory mixture, while the extract 7 was found ameliorative only in this pro-inflammatory context (FIG. 10).

It is not technically possible to perform the same assay on the colonocyte populations, as they do not replicate the intestinal barrier in vitro.

From the analysis of the differentiated cells (enterocytes), the extract 7 (mix CBD/CBG 5:2.5%; absolute Ethanol extract) is the one with the greatest protection of the epithelial barrier, together with a significant reduction of CXCL10.

Following the first experiments carried out to complete the experimentation Phase 1 illustrated in figures from 1 to 11, the inventors of the present invention have selected the extracts 5 (Mix CBD/CBG 5:5%; 100% ethanol extract) and 6 (Mix CBD/CBG 5:5%; 50% ethanol extract) to proceed with Phase 2 and Phase 3 of the experimentation which will be specifically described below. The extracts 5 and 6 come from the same raw materials and have the same concentration in main cannabinoids (CBG and CBD) but have been treated differently during the extraction phase.

### Phase 2: Gastrointestinal Digestion of the Extracts Selected in Phase 1 and Evaluation of Stability in the Gastrointestinal Environment

The extracts 5 and 6 were subjected to simulated gastro-intestinal digestion, which involves changing the pH, adding enzymes typical of the gastric (pepsin) and intestinal phase (lipase, trypsin, pancreatin, bile salts) and agitation, aimed at simulating peristalsis.

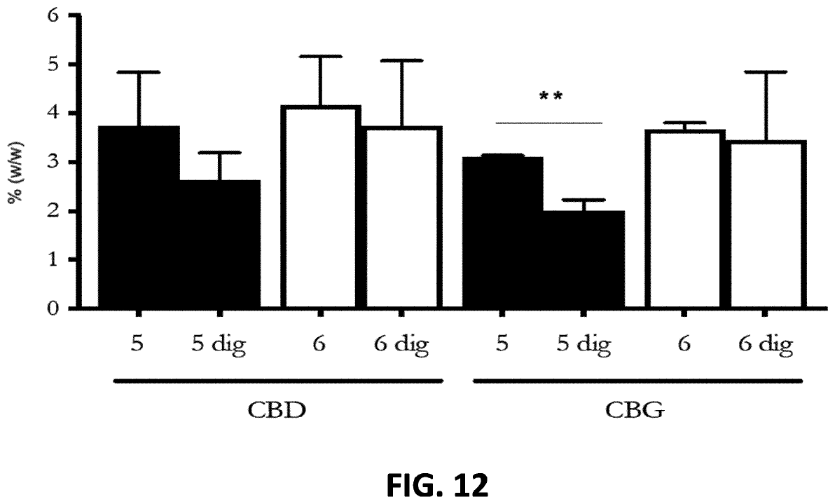

Following the digestive process, CBD and CBG were quantified by LC-MS, in a comparison with the original undigested extracts (FIG. 12).

The results show that in the extract 5 there is a significant reduction in the cannabinoid titre only for CBG, while for CBD there is a non-significant decrease. The extract 6 shows no difference in the two phytocannabinoids following simulated digestion.

The extract 6 is more resistant to the digestion process than the extract 5, obtained with a different method but coming from the same raw materials.

### Phase 3: Study of the Anti-Inflammatory Activity and the Effect of the Extracts on Paracellular Permeability

In the first phase of the work, the combination of IL-1β (10 ng/ml) and IFN-γ (10 ng/mL) was selected as pro-inflammatory stimulation. During the third experimental phase, the cytotoxicity of the extracts in both colonocytes and enterocytes was compared, using this pro-inflammatory cocktail.

The MTT assay did not highlight a reduction in viability following treatment with the extracts 5 and 6 in the colonocytes or in the enterocytes.

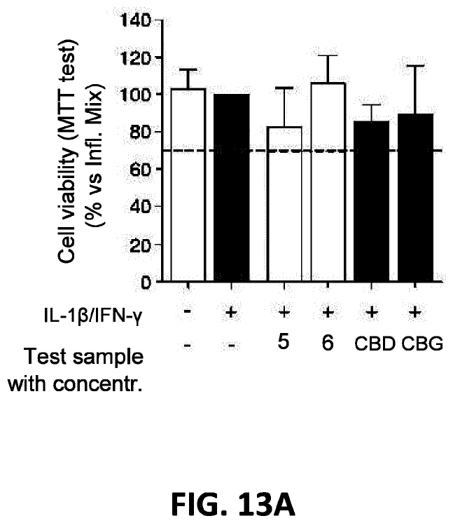

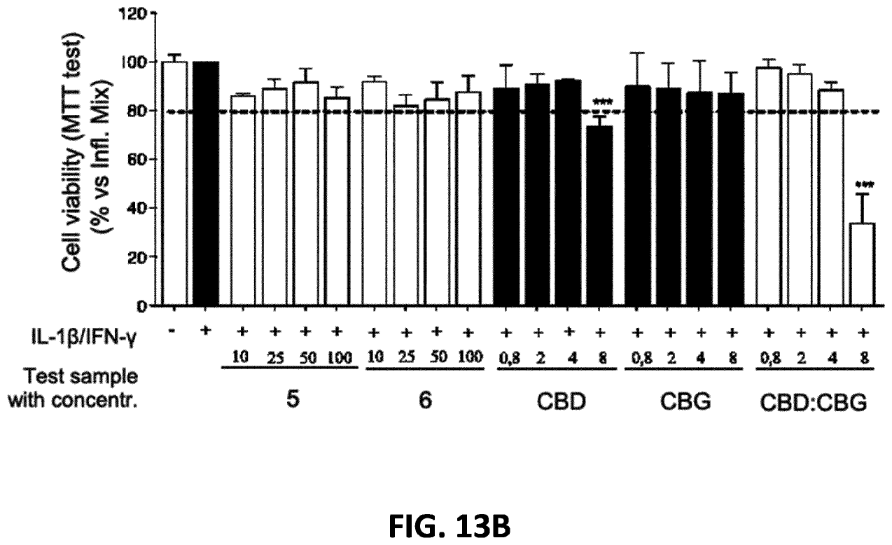

In contrast, only in the colonocytes, a significant reduction was observed following treatment with CBD at the highest concentration assayed (8 μM, −25%) or with the combination CBD (8 μM)+CBG (8 μM, −70%) (FIG. 13A and B).

The extracts subjected to simulated digestion were used in the subsequent experimental phase, aimed at determining their anti-inflammatory activity in the colonocyte and enterocyte.

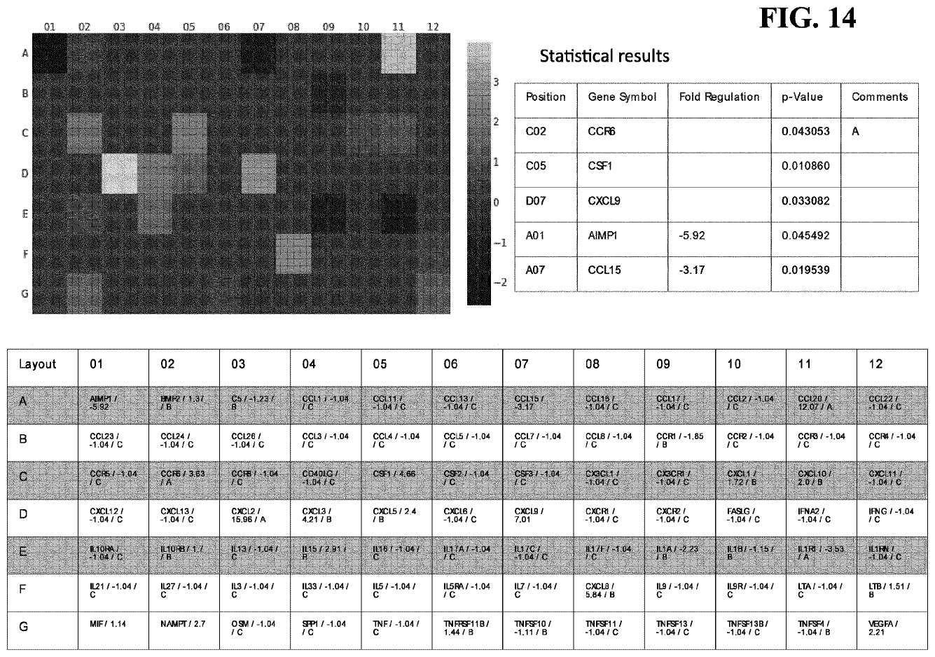

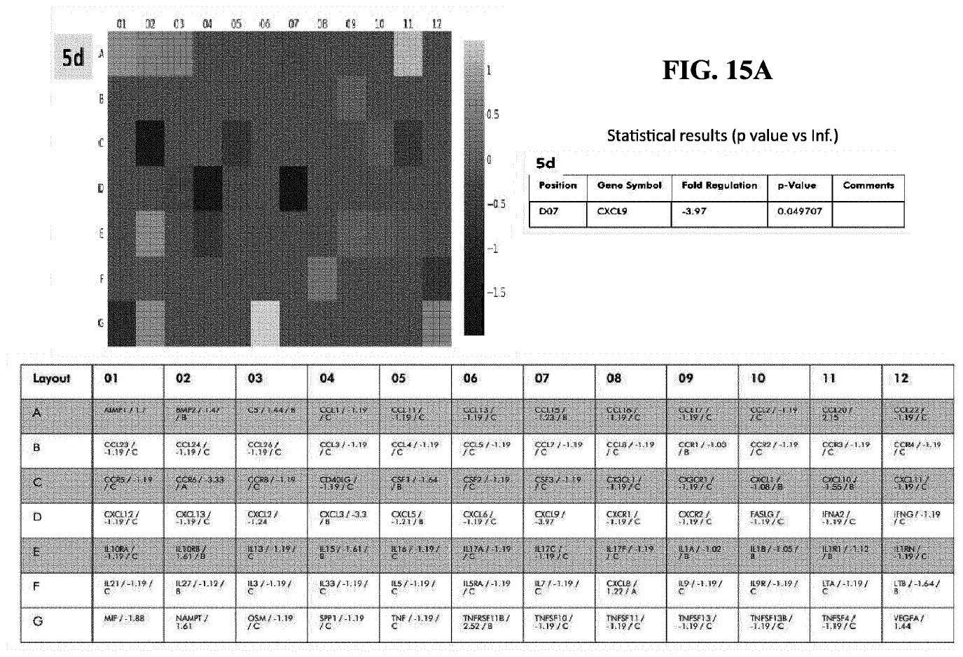

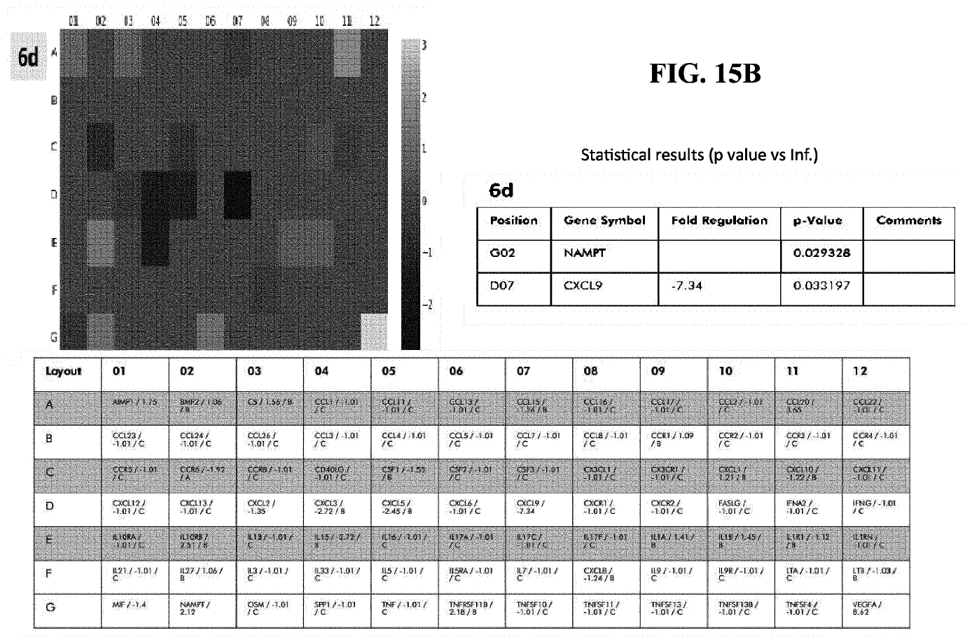

The pro-inflammatory response induced by IL-Iβ (10 ng/ml) and IFN-γ (10 ng/mL) in the colonocyte is characterized by increased transcription of chemokines and receptors which are important m chronic inflammation, such as CCL20 (Macrophage Inflammatory Protein-3), CXCL2 (Macrophage Inflammatory protein 2-alpha), CSF1 (Colony Stimulating Factor 1), CXCL9 and CXCL10 (chemokines for macrophages and lymphocytes induced by IFN-γ), IL15, CCR6 (receptor for CCL20) (FIG. 14). The extracts subjected to digestion (5d, 6d) generally reduced the expression of the inflammatory genes with a similar profile to each other, but the inhibitory effect was found significant specifically for CXCL9 (FIG. 15A,15B), a chemokine that attracts lymphocytes, typical of the inflammatory bowel processes.

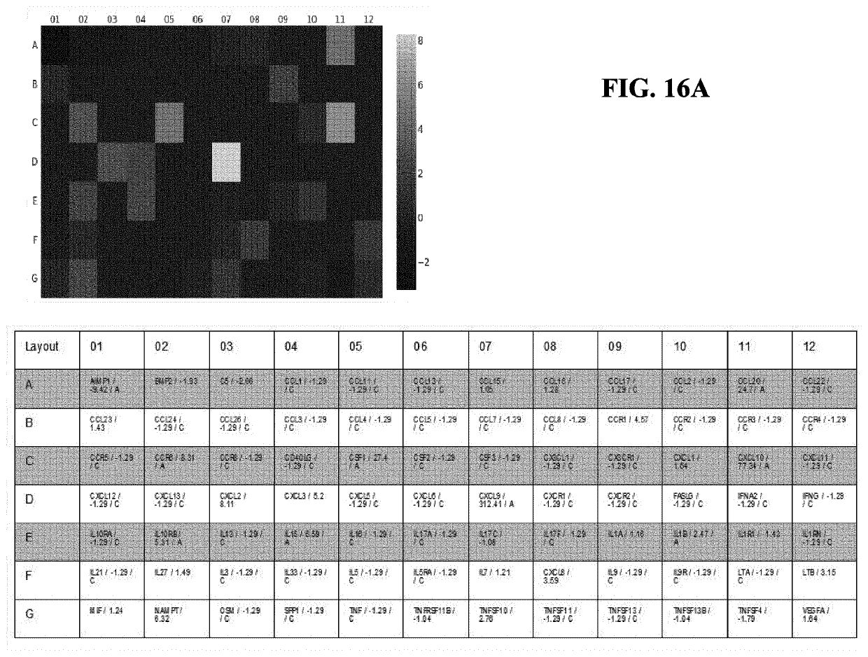

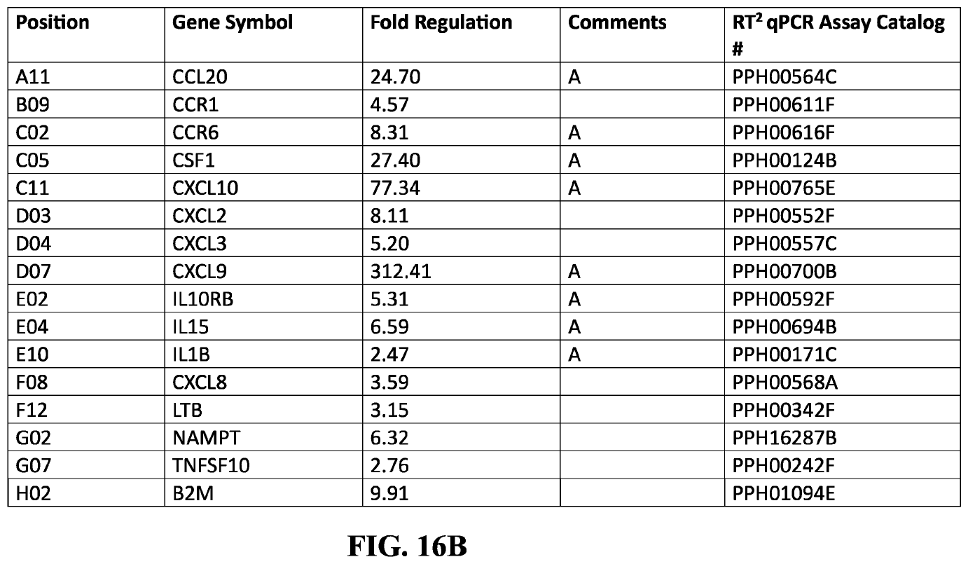

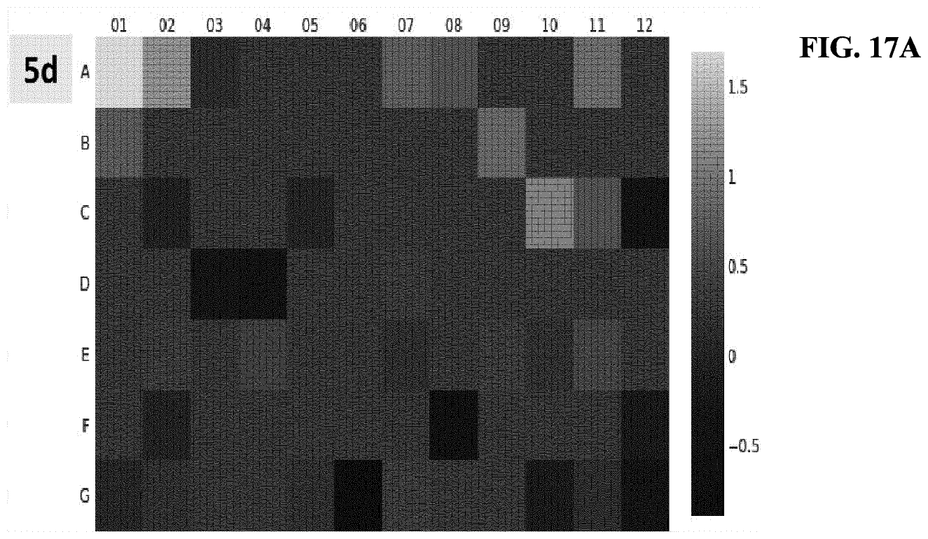

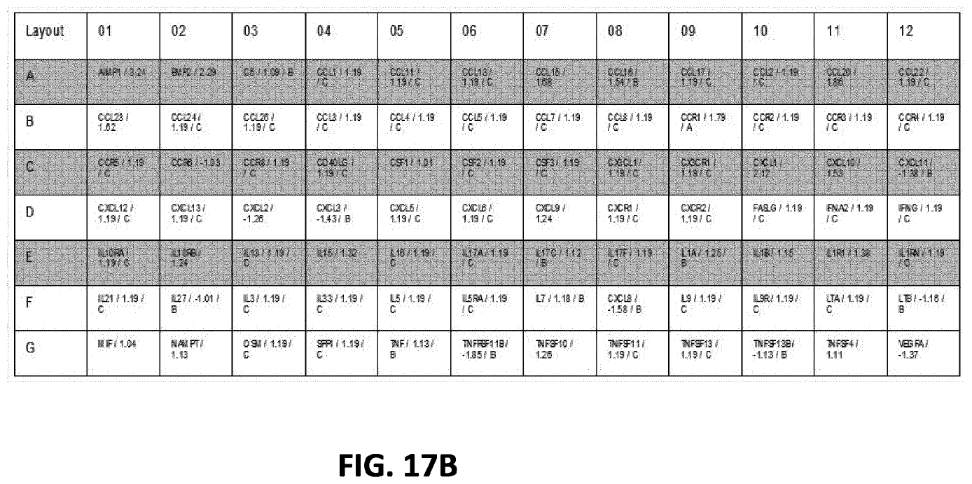

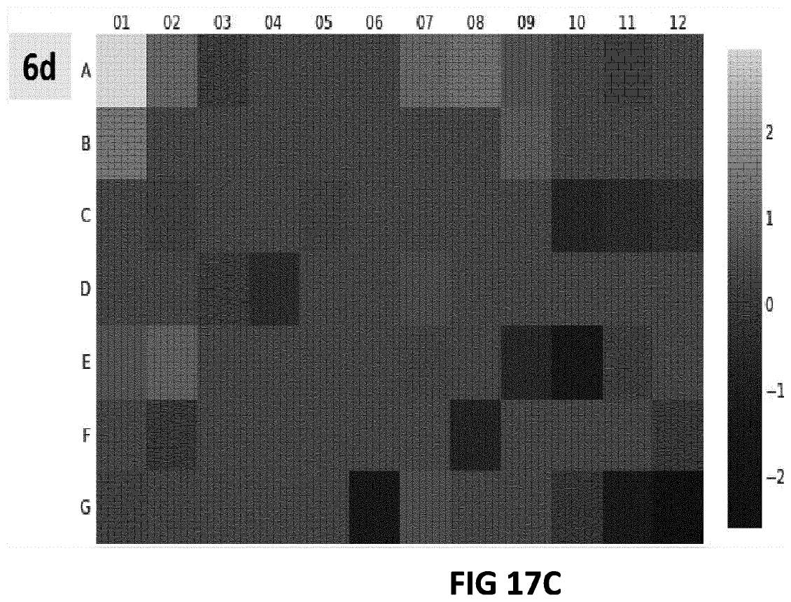

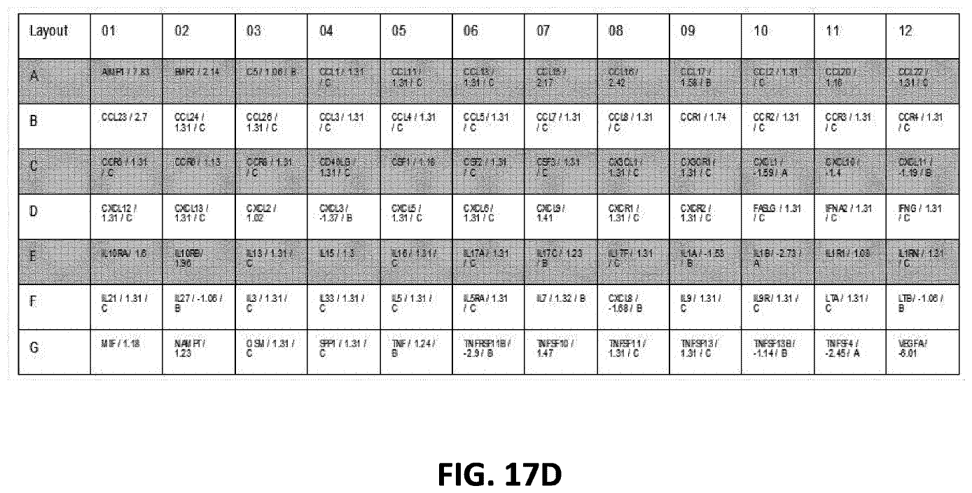

The same transcriptional evaluation was performed in differentiated colonocytes on Transwell® support (enterocyte). The pro-inflammatory response induced by IL-1β (10 ng/mL) and IFN-γ (10 ng/mL) was found comparable to that obtained in the colonocyte: in particular, attractive chemokine genes were more transcribed for macrophages and lymphocytes (CCL-20, CXCL-2, CXCL-3, CXCL-9, CXCL-10) and, to a lesser extent, also for interleukins (IL-15, IL-1B) (FIG. 16A,16B). In this case, the extracts did not significantly change the transcriptional profile (FIG. 17A, 17B, 17C, 17D), in line with what was observed in Phase 1, i.e. in the experiments relative to the release of inflammatory mediators.

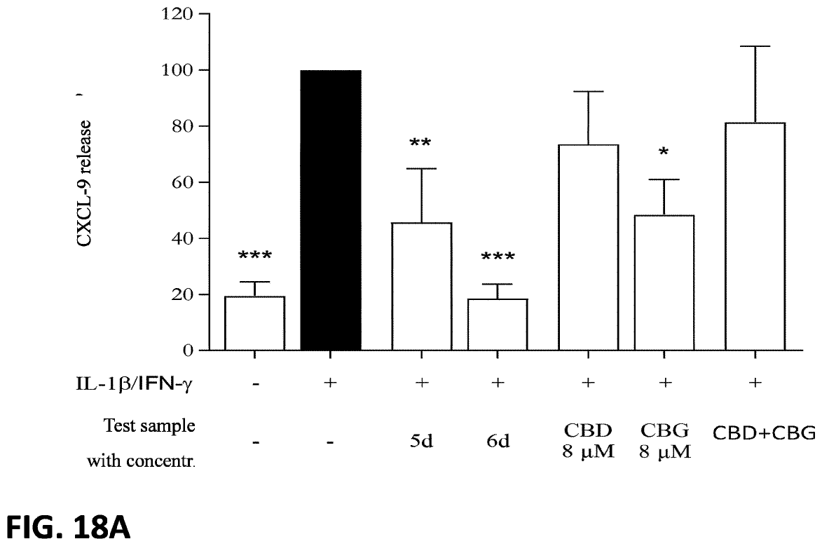

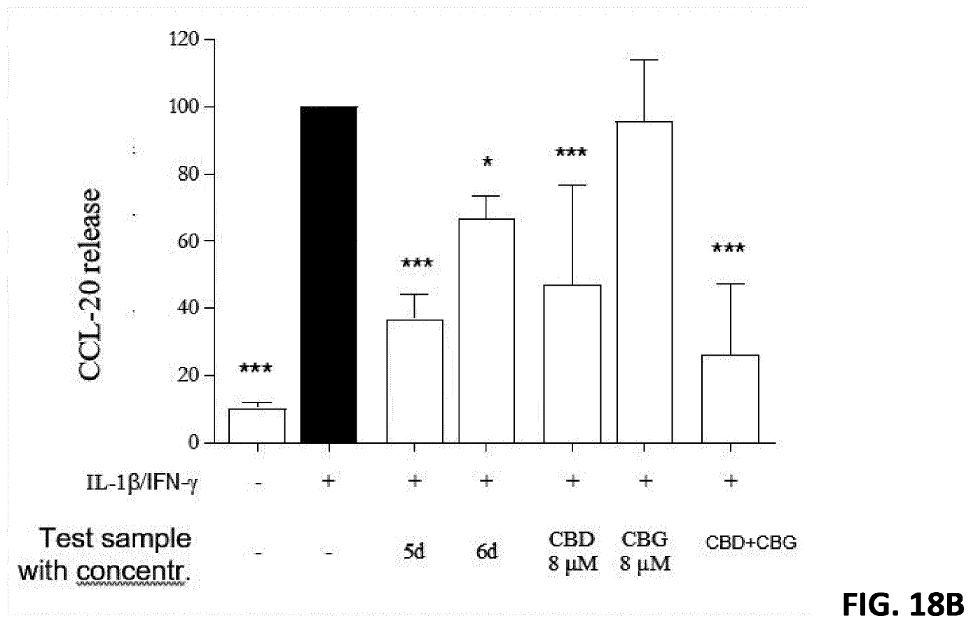

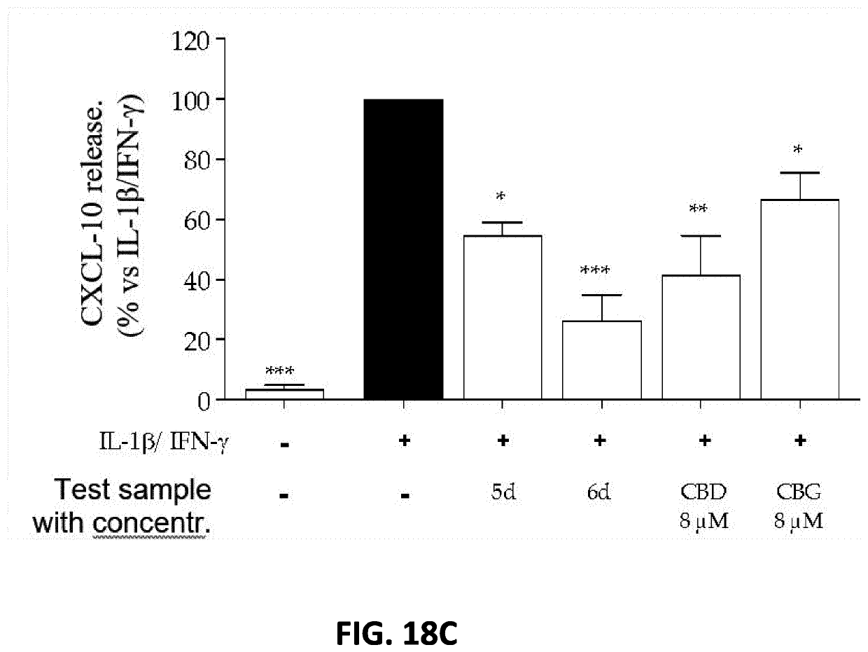

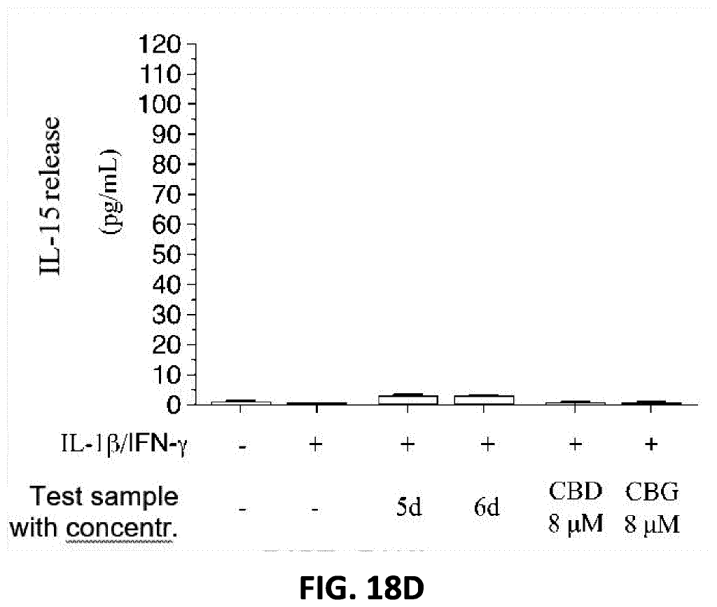

Consequently, the effect of the extracts at the protein level, especially in the colonocyte, was evaluated by ELISA assays for inflammatory mediators selected starting from the transcriptional data. All mediators are regulated by NF-κB. One more time, both extracts significantly inhibited CXCL-9 release, which emerged as a relevant marker from the transcription analyses (FIG. 18A, 18B, 18C, 18D). In addition, inhibitory activity on the release of CCL-20, as an additional highly transcribed mediator, as well as the inhibition of CXCL-10, a mediator measured in the first screening phase, was demonstrated. Finally, despite the increase at the transcriptional level, IL-15 was not significantly produced at the protein level.

For some mediators, in addition to the individual cannabinoids, the role of their 1:1 combination (8 μM) has been studied. It is interesting to note that CBD or CBG, considered individually, can inhibit different inflammatory mediators; consequently, in the extracts in which they are in admixture, they maintain their activity and confer a non-specific inhibitory action. In particular, CBG appears to participate in the inhibitory activity towards IFN-γ-sensitive chemokines (CXCL-9, CXCL-10), while CBD has a more or less marked inhibitory effect on all the mediators.

Although their combination also explains in some cases the inhibitory effect (CCL-20), they were attributed a certain cytotoxicity in the preliminary experiments, when considered separately from the source extract.

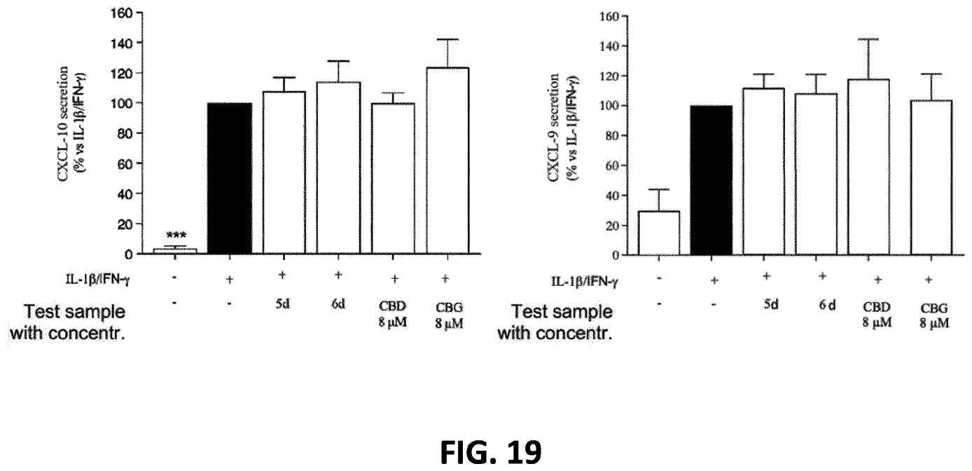

In line with what was observed in the previous results, the extracts did not inhibit the release of inflammatory mediators in the enterocyte model (FIG. 19). The same was highlighted with the use of pure cannabinoids. In analogy with the colonocyte, the release of IL-15 was found not relevant in the enterocyte model, compared to an increase at the transcriptional level.

In addition to anti-inflammatory effects, the first experimental phase had demonstrated a mild protective effect on the epithelial integrity by the selected extracts. Their activity was re-evaluated following the simulation of the digestive process, but it was also further investigated in a second experimental model, in which the involvement of the macrophage (THP-1 line) in co-culture with the enterocyte is envisaged.

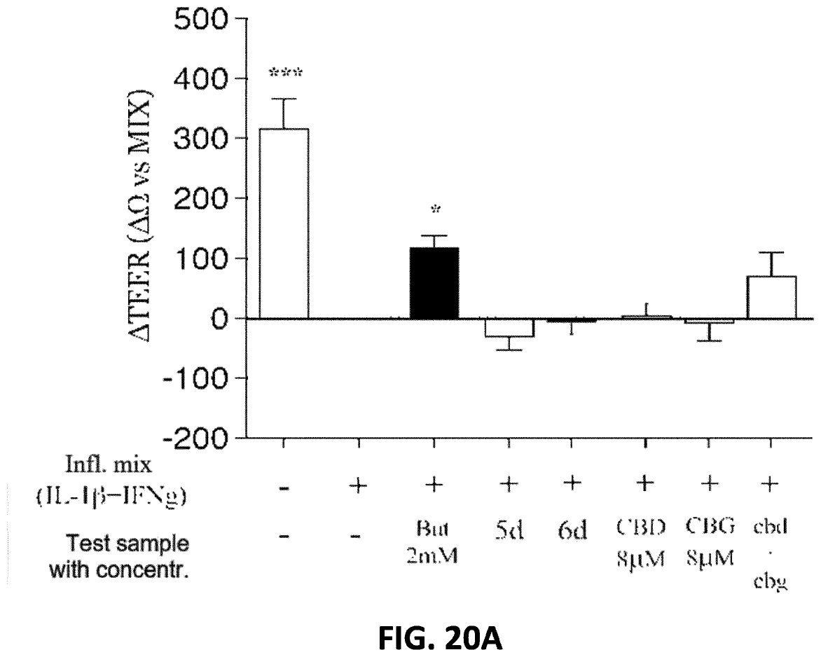

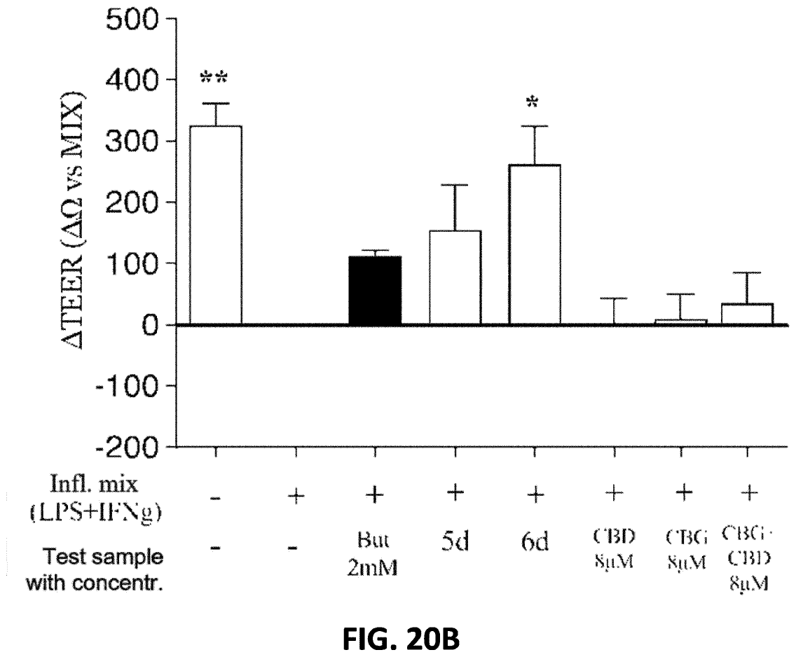

The extracts and the relative cannabinoids did not result in the restoration of IL-1β- and IFN-γ-induced paracellular permeability in the enterocyte; on the contrary, they led to a partial restoration in the enterocyte-macrophage co-culture model, a significant result for the extract 6 (FIG. 20A and B).

The effect was not attributed to CBD, CBG or their combination.

It is likely that the protective activity is linked to an anti-inflammatory effect on the macrophage, which indirectly affects the restoration of the epithelial integrity. Furthermore, it cannot be excluded that CBD and CBG, known for their immunosuppressive activities, can effectively permeate the intestinal epithelium and reach the macrophage thanks to adjuvant compounds present in the extract, but not in pure form.

The result was also corroborated by the evaluation of the paracellular passage of a fluorescent probe (Lucifer Yellow, LY) through the intestinal epithelium. In addition, the effect towards the expression of proteins of the intercellular junctions (occludin and zonulin 1) was evidenced by immunofluorescence.

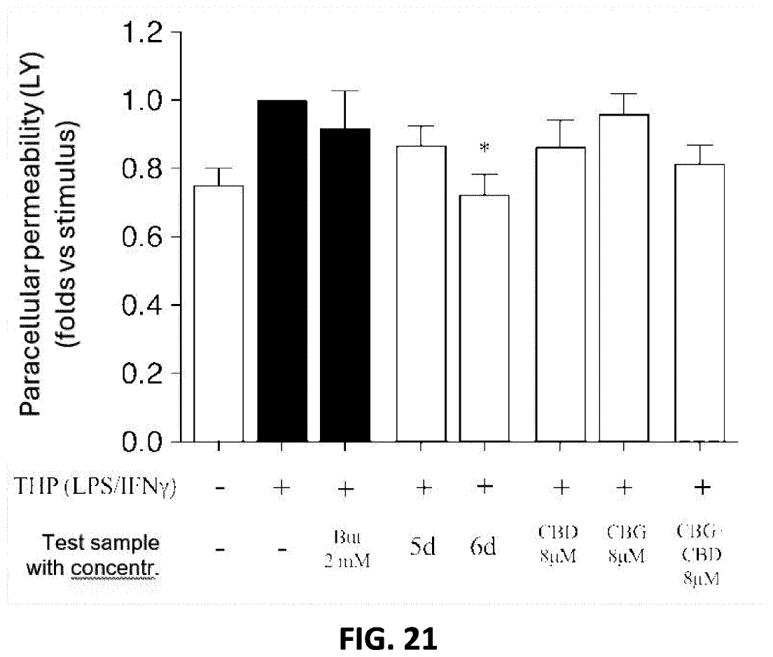

The results confirm the significant effect of restoration of the paracellular integrity by the extract 6 in the co-culture model (FIG. 21).

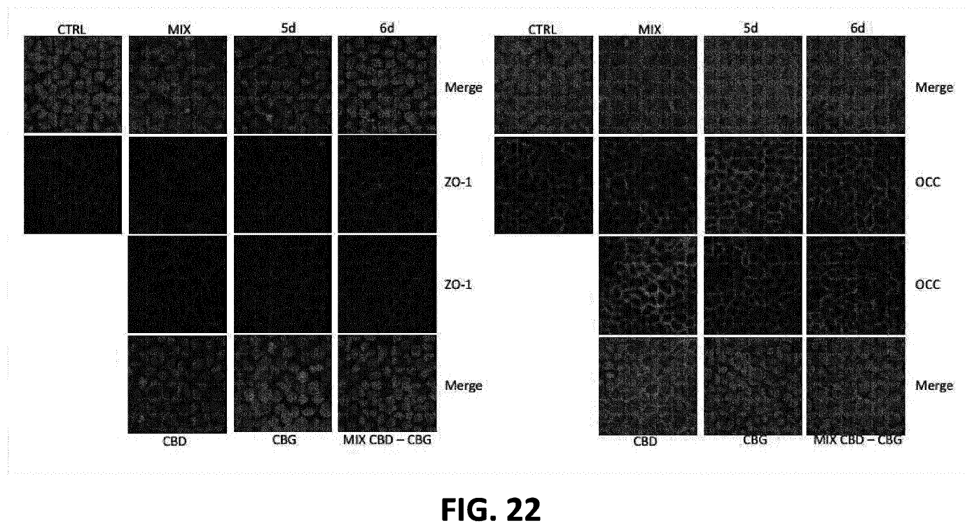

The same extract also clearly restored zonulin-1 (ZO-1) expression and organization, while both extracts increased occludin (OCC) expression (FIG. 22).

In FIG. 20B, the synergistic effect due to the use of an extract, in particular the extract 6 (Mix CBD/CBG 5:5%), which has a surprisingly high efficacy compared to the efficacy of the isolated CBD and/or CBG components at the same concentration as the extract 6, can be clearly noted.

Advantageously, furthermore the selected cannabinoids do not present, compared to other cannabinoids such as THC, the risk of onset, at certain concentrations, of psychotropic effects that may limit their therapeutic use. In particular, the extracts subject-matter of the invention have a concentration of THC, CBN, THCV always significantly below 0.2% and therefore well below the concentrations from which narcotic effects are triggered.

### Phase 4: Prebiotic Activity

In the last phase of the project, the prebiotic effect of the extracts 5 and 6 towards enteric bacterial strains was evaluated by means of fecal fermentation followed by gene analysis of selected strains.

Following fermentation of the extracts, the level of butyric acid produced by the intestinal flora was measured as an indicator of SCFA production. The reliability of the quantitative analysis was reinforced by two different techniques, namely an ELISA assay and mass spectrometry.

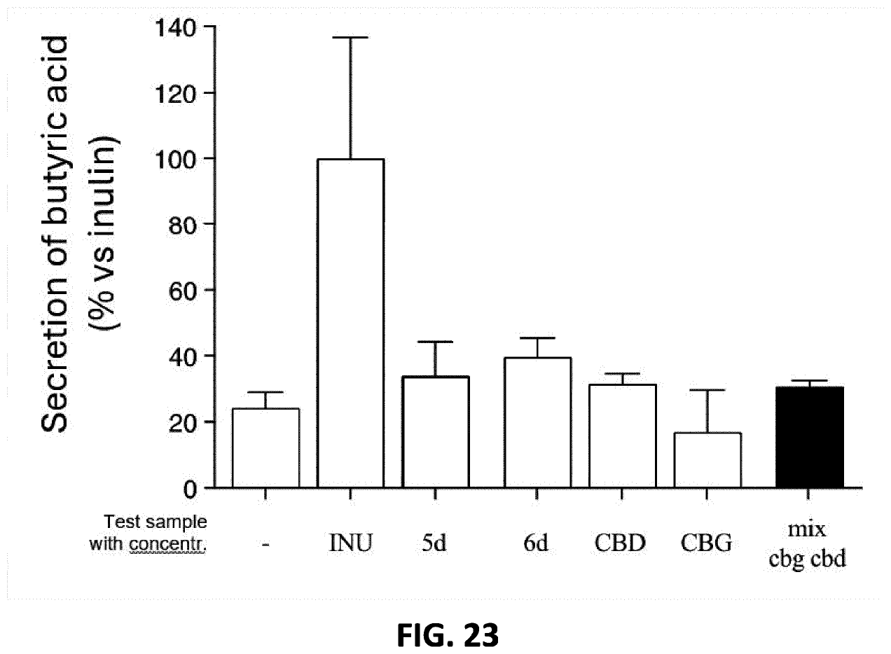

The second was carried out in collaboration with the UNITECH-OMICS technology platform. The results of the ELISA assay (FIG. 23) indicate that the extract 5 and 6 do not significantly induce an increase in butyric acid levels, compared to the reference prebiotic inulin (increase of 5 times).

Table 2 below reports the prebiotic effect of the extracts 5 and 6 subjected to intestinal digestion (5d, 6d) and of the cannabinoids (CBD, CBG) in terms of induction of butyric acid (SCFA) production by the human microbiota of fecal origin. The result is expressed in μg/mL (LC-MS analysis).

TABLE 2

| | | | μg/ml | | |
|---|---|---|---|---|---|
| | | Dilution | Mean | | |
| Sample | Sample description | factor | (n = 4) | ±SD | CV % |
| Sample 1 | Fecal pool alone, | 5 | 276.48 | 29.458 | 10.7 |
| | Blank (absence of | | | | |
| | molecules/extracts) | | | | |
| Sample 2 | Inulin, positive | 50 | 638.87 | 12.188 | 1.9 |
| | control | | | | |
| Sample 3 | Extract 6 | 50 | 61.23 | 0.616 | 1.0 |
| Sample 4 | Extract 5 | 50 | 69.42 | 0.636 | 0.9 |
| Sample 5 | CBD, Cannabidiol | 5 | 212.20 | 33.912 | 16.0 |
| Sample 6 | CBG, Cannabigerol | 5 | 173.54 | 18.762 | 10.8 |
| Sample 7 | MIX CBD:CBG, | 5 | 171.63 | 16.629 | 9.7 |
| | combination CBD | | | | |
| | and CBG 1:1 | | | | |

The absence of negative effects on the bacterial populations considered can be observed. The data does not provide information about the activity of the two extracts but measures their intestinal tolerability.

### CONCLUSIONS

The results obtained in this project (Phase 1, 2, 3, 4) allow to make some considerations on the extracts under study and the pure molecules.

The extracts 5 and 6 report a better cytotoxicity profile, i.e. they have no cytotoxic effect at the highest concentration assayed (100 μg/mL), compared to isolated CBD and its combination with CBG.

Both extracts have anti-inflammatory activity in the colonocyte, both at the transcriptional level, and at the level of release of inflammatory mediators, but not in the enterocyte. The activity is mainly attributable to CBD, but CBG can also explain the inhibition of some mediators (CXCL-9, CXCL-10) typical of intestinal inflammation.

The extract 6 has a higher protective activity on the epithelial integrity than the reference butyric acid (2 mM). The mechanism of action could be linked to the anti-inflammatory effects on the macrophage.

The activity was not attributed to CBD or CBG, which never showed inhibitory effects in the enterocyte at the concentrations assayed.

The composition comprising at least one cannabis extract for use in the treatment of an inflammatory bowel disease and/or in the prevention of bowel diseases correlated to chronic inflammation and dysbiosis, is susceptible to numerous modifications and variants all falling within the scope of the inventive concept described and claimed.

1. A composition comprising a blend of at least two standardized extracts in their active components coming from a stable variety of Cannabis sativa L., for use in the treatment of an inflammatory bowel disease and/or in the prevention of bowel diseases correlated to acute, chronic inflammation and dysbiosis, said standardized extracts comprising
in their active components cannabigerol and cannabidiol in various mass ratios.

2. The composition according to claim 1, wherein said mass ratios are chosen from 1:1, 1:2 or 2:1.

3. The composition accord to claim 1, wherein the cannabidiol and cannabigerol, are each provided in a concentration equal to 5% by weight.

4. The composition according to claim 1, wherein the cannabidiol and cannabigerol, respectively, are provided in concentrations equal to 5% and 2.5% by weight or vice versa.

5. The composition according to claim 1, further comprising an MCT oil-based carrier.

6. The composition according to claim 1, wherein said inflammatory bowel disease is selected from the group comprising: chronic intestinal inflammations, leaky-gut syndrome, dysbiosis, celiac disease, intestinal inflammation of bacterial or parasitic origin, diverticulitis and irritable bowel syndrome.

7. The composition according to claim 6, wherein said chronic intestinal inflammation comprises ulcerative colitis and Crohn's disease.

8. The composition comprising at least one cannabis extract according to claim 1 for use in reducing the secretion of CXCL10 involved in an inflammatory bowel disease.
